(19) United States
(12) Patent Application Publication
(10) Pub. No.: US 2010/0152285 A1
(43) Pub. Date: Jun. 17, 2010

(54) Flavononol Renin Inhibitor Compounds and Methods of Use Thereof

(75) Inventors: Randall S. Alberte, Estero, FL (US); William P. Roschek, JR., Naples, FL (US)

Correspondence Address: FOLEY HOAG, LLP;PATENT GROUP, WORLD TRADE CENTER WEST, 155 SEAPORT BLVD, BOSTON, MA, 02110, US

(73) Assignee: HerbalScience Group LLC, Naples, FL (US)

(21) Appl. No.: 12/639,437

(22) Filed: Dec. 16, 2009

Related U.S. Application Data
(60) Provisional application No. 61/122,836, filed on Dec. 16, 2008.

(51) Int. Cl. A61K 31/353; A61P 13/10; A61P 13/12; A61P 9/10

(52) U.S. Cl. 514/456

(57) ABSTRACT

The present disclosure relates in part to methods of treating or preventing renin mediated conditions comprising administering to a subject in need thereof a flavononol compound represented by formula I:

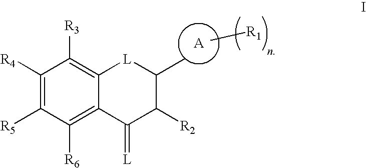

Another aspect of the invention is a method for treating or preventing cardiovascular diseases such as high blood pressure, and maintaining electrolyte homeostasis and proper renal function in a subject by administering the compounds of Formula I to the subject.

### RELATED APPLICATIONS

This application claims the benefit of priority to U.S. Provisional Application No. 61/122,836, filed on Dec. 16, 2008, the contents of which are hereby incorporated in their entirety.

### FIELD OF INVENTION

The present invention relates to flavononols and related molecules that inhibit the renin enzyme. These inhibitors are useful for treating or preventing hypertension, high blood pressure, diabetic nephropathy, and other cardiovascular diseases involving vasoconstriction.

### BACKGROUND OF THE INVENTION

Renin is an enzyme that plays a critical role in the maintenance of blood pressure and electrolyte homeostasis in the body and is a key component of the Renin-Angiotensin System (RAS) (N. Basso and N. A. Terragno, 2001. History About the Discovery of the Renin-Angiotensin System, Hypertension. 38:1246-1249; J. E. Hall, 2003. Historical perspective of the renin-angiotensin system, Mol. Biotechnol. 24:27-39; F. J. He and G. A. MacGregor, 2003. Salt, blood pressure and the renin-angiotensin system, J Renin Angiotensin Aldosterone Syst. 4:11-16; J. L. Lavoie and C. D. Sigmund, 2003. Minireview: Overview of the Renin-Angiotensin System—An Endocrine and Paracrine System, Endocrinology. 144:2179-2183). Renin production in the kidneys is initiated when blood pressure is low (E. Hackenthal, M. Paul, D. Ganten and R. Taugner, 1990. Morphology, physiology, and molecular biology of renin secretion, Physiol. Rev. 70:1067-1116). Renin converts the liver product Angiotensinogen to Angiotensin I (AI) (C. F. Deschepper, 1994. Angiotensinogen: hormonal regulation and relative importance in the generation of angiotensin II, Kidney Int. 46:1561-1563). Although AI is not believed to be the major signaling molecule for blood pressure maintenance, it is the substrate for Angiotensin Converting Enzyme (ACE2) that produces Angiotensin II (AII) (J. E. Hall, 2003. Historical perspective of the renin-angiotensin system, Mol Biotechnol. 24:27-39; F. J. He and G. A. MacGregor, 2003. Salt, blood pressure and the renin-angiotensin system, J Renin Angiotensin Aldosterone Syst. 4:11-16; J. L. Lavoie and C. D. Sigmund, 2003. Minireview: Overview of the Renin-Angiotensin System—An Endocrine and Paracrine System, Endocrinology. 144:2179-2183). Angiotensin II constricts blood vessels, which increases blood pressure, and increases the production of Aldosterone and Vasopressin leading to increased sodium and water retention. Since Renin is a key component of RAS, inhibitors of this enzyme represent a potential treatment for high blood pressure. Currently, Aliskiren, a known Renin Inhibitor, has been approved by the FDA for treatment of hypertension (J. M. Wood, J. Maibaum, J. Rahuel, M. G. Grutter, N. C. Cohen, V. Rasetti, H. Ruger, R. Goschke, S. Stutz, W. Fuhrer, W. Schilling, P. Rigollier, Y. Yamaguchi, F. Cumin, H. P. Baum, C. R. Schnell, P. Herold, R. Mah, C. Jensen, E. O'Brien, A. Stanton and M. P. Bedigian, 2003. Structure-based design of aliskiren, a novel orally effective renin inhibitor, Biochem Biophys Res Commun. 308:698-705; A. H. Gradman, R. E. Schmieder, R. L. Lins, J. Nussberger, Y. Chiang and M. P. Bedigian, 2005. Aliskiren, a novel orally effective renin inhibitor, provides dose-dependent antihypertensive efficacy and placebo-like tolerability in hypertensive patients, Circulation. 111:1012-1018).

More recently low-dose spironolactone has been shown to reduce blood pressure when combined with ACE inhibitors (A. S. Bomback, P. Muskala, E. Bald, G. Chwatko and M. Nowicki, 2009. Low-dose spironolactone added to long-term ACE inhibitor therapy reduces blood pressure and urinary albumin excretion in obese patients with hypertensive target organ damage, Clin. Nephrol. 72:449-456). Recent evidence shows that angiotensin receptor blockers (ARB) have similar efficacy to ACE inhibitors in reducing cardiovascular outcomes and that combination therapy has been shown to be beneficial in patients with congestive heart failure or renal disease (C. Schindler, 2008. ACE-inhibitor, AT1-receptor-antagonist, or both? A clinical pharmacologist's perspective after publication of the results of ONTARGET, Ther. Adv. Cardiovasc. Dis. 2:233-248; B. S. Heran, M. M. Wong, I. K. Heran and J. M. Wright, 2008. Blood pressure lowering efficacy of angiotensin receptor blockers for primary hypertension, Cochrane Database Syst. Rev. 4:CD003822; A. Ravandi and K. K. Teo, 2009. Blocking the renin-angiotensin system: dual-versus mono-therapy, Expert Rev. Cardiovasc. Ther. 7:667-674). More effective and safer ACE inhibitors suitable for co-therapy and mono-therapy are needed to address the more complex needs in cardiovascular health.

The present invention provides in part improved inhibitors of the Renin enzyme based on identified bioactives that have demonstrated Renin inhibition activity.

### SUMMARY

One aspect of the invention relates to a method of treating or preventing a renin-mediated condition in a subject in need thereof comprising administering to the subject a therapeutically effective amount of a compound of formula I:

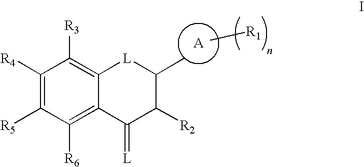

or a pharmaceutically acceptable salt thereof,
wherein, independently for each occurrence:

R₁ represents alkoxy, alkenyloxy, alkynyloxy, aryloxy, arylalkyloxy, hydroxy, —OC(O)—R₇, alkyl, alkenyl, alkynyl, acetyl, formyl, halide, cyano, nitro, SH, amino, amido, sulfonyl, or sulfonamido;

R₂ represents —OH or

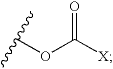

R₃, R₄, R₅, and R₆ represent H, hydroxy, alkoxy, alkenyloxy, alkynyloxy, aryloxy, aralkyloxy; —OC(O)—R₇, alkyl, alkenyl, alkynyl, aralkyl, acetyl, formyl, halide, cyano, nitro, SH, amino, amido, sulfonyl, or sulfonamido;

R₇ represents H, alkyl, alkenyl, alkynyl, cycloalkyl, aryl, arylalkyl or a carbohydrate;

A represents an aryl group;

L represents O, S, or NR;

R represents H, hydroxy, alkyl, alkenyl, alkynyl, aralkyl, acetyl, formyl, or sulfonyl;

X represents a carbohydrate, a cycloalkyl, or a cycloalkenyl group; and

n represents an integer from 1 to 5, inclusive;

wherein any of the aforementioned alkoxy, alkenyloxy, alkynyloxy, aryloxy, aralkyloxy, alkyl, alkenyl, alkynyl, cycloalkyl, cycloalkenyl, aryl and aralkyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

In some embodiments, R₁ represents H, alkoxy, aryloxy, aralkyloxy, hydroxy, —OC(O)—R₇, alkyl, acetyl, formyl, or halide;

R₂ represents

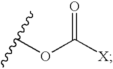

R₃, R₄, R₅, and R₆ represent H, alkoxy, aryloxy, aralkyloxy; —OC(O)—R₇, alkyl, aralkyl, acetyl, formyl, or halide;

R₇ represents H, alkyl, aryl, or arylalkyl;

A represents an aryl group;

L represents O;

X represents a carbohydrate, a cycloalkyl, or a cycloalkenyl group; and

n represents an integer from 1 to 5, inclusive;

wherein any of the aforementioned alkoxy, alkenyloxy, alkynyloxy, aryloxy, aralkyloxy, alkyl, alkenyl, alkynyl, aryl, aralkyl, cycloalkyl and cycloalkenyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

In some embodiments, the carbohydrate is selected from the group consisting of a monosaccharide, a disaccharide, an oligosaccharide, and a polysaccharide.

In other embodiments, R₂ is —OH.

In other embodiments, L is O.

In other embodiments, R₃, R₄, R₅ and R₆ are each independently H or hydroxy, wherein at least two of R₃, R₄, R₅ and R₆ are hydroxy.

In other embodiments, R₁ is hydroxy, and n is equal to 2 or 3.

In other embodiments, A is a benzene ring.

In other embodiments, X is a carbohydrate.

In other embodiments, X is a cycloalkyl or cycloalkenyl group; and wherein the cycloalkyl or cycloalkenyl group is substituted with 1 to 3 hydroxy groups.

Another aspect of the invention relates to a method of treating or preventing a renin-mediated condition in a subject in need thereof comprising administering to the subject a therapeutically effective amount of a compound of formula Ia:

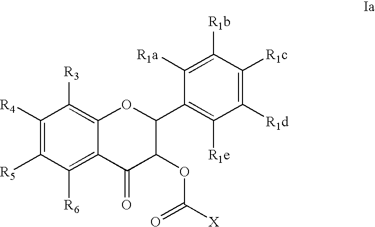

or a pharmaceutically acceptable salt thereof,
wherein, independently for each occurrence:

R₁ₐ, R_{1b}, R_{1c}, R_{1d}, R₁ₑ represent H, hydroxy, alkoxy, aralkyloxy, or aryloxy;

R₃, R₄, R₅, and R₆ represent H, hydroxy, alkoxy, aryloxy, or aralkyloxy; and

X represents a carbohydrate, a cycloalkyl, or a cycloalkenyl group;

wherein any of the aforementioned alkoxy, aryloxy, aralkyloxy may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

In some embodiments, independently for each occurrence:

R₁ₐ, R_{1b}, R_{1c}, R_{1d}, and R₁ₑ represent H, hydroxy, alkoxy, aralkyloxy, or aryloxy; provided that at least two of R₁ₐ, R_{1b}, R_{1c}, R_{1d}, and R₁ₑ are hydroxy;

R₃, R₄, R₅, and R₆ represent H, hydroxy, alkoxy, aryloxy, or aralkyloxy, provided that at least two of R₃, R₄, R₅, and R₆ are hydroxy; and

X is carbohydrate, cycloalkyl, or cycloalkenyl;

wherein any of the aforementioned alkoxy, aryloxy, aralkyloxy, cycloalkyl, or cycloalkenyl may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

In other embodiments, R₁ₐ, R_{1b}, R_{1c}, R_{1d}, and R₁ₑ represent H or hydroxy, and three of R₁ₐ, R_{1b}, R_{1c}, R_{1d}, and R₁ₑ are hydroxy.

In other embodiments, R₃, R₄, R₅, and R₆ represent H or hydroxy, and two of R₃, R₄, R₅, and R₆ are hydroxy.

In other embodiments, X is a carbohydrate selected from the group consisting of a monosaccharide, a disaccharide, an oligosaccharide, and a polysaccharide.

In other embodiments, X is a carbohydrate selected from the group consisting of arabinose, lyxose, ribose, rhamnose, deoxyribose, xylose, ribulose, xylulose, allose, altrose, galactose, glucose, gulose, idose, mannose, talose, fructose, psicose, sorbose, tagatose, sucrose, lactose, maltose, trehalose or cellobiose, raffinose, maltodextrin, cyclodextrin, starch, glycogen, dextran, and cellulose.

In other embodiments, X is rhamnose.

In other embodiments, X is a cycloalkyl or cyloalkynyl group, wherein the cycloalkyl or cycloalkenyl group is substituted with one to three hydroxy groups.

Another aspect of the invention relates to a method of treating or preventing a renin-mediated condition in a subject in need thereof comprising administering to the subject a therapeutically effective amount of a compound of formula Ib:

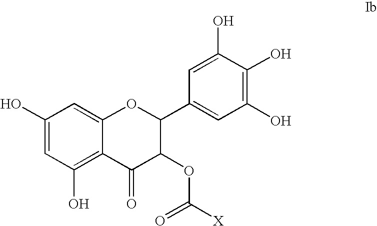

or a pharmaceutically acceptable salt thereof,
wherein X represents a carbohydrate, a cycloalkyl, or a cycloalkenyl, wherein the cycloalkyl or cycloalkenyl may be substituted with one to three hydroxy groups.

In some embodiments, X is a carbohydrate selected from the group consisting of arabinose, lyxose, ribose, rhamnose, deoxyribose, xylose, ribulose, xylulose, allose, altrose, galactose, glucose, gulose, idose, mannose, talose, fructose, psicose, sorbose, tagatose, sucrose, lactose, maltose, trehalose, cellobiose, raffinose, maltodextrin, cyclodextrin, starch, glycogen, dextran, and cellulose.

In other embodiments, X is a cyclohexyl or cyclohexenyl substituted with 1 to 3 hydroxy groups.

In other embodiments, X is:

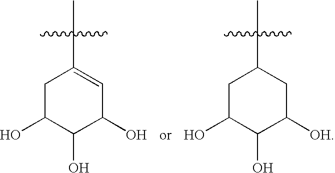

Another aspect of the invention relates to a method of treating or preventing a rennin-mediated condition in a subject in need thereof comprising administering to the subject a therapeutically effective amount of a compound selected from the group consisting of:

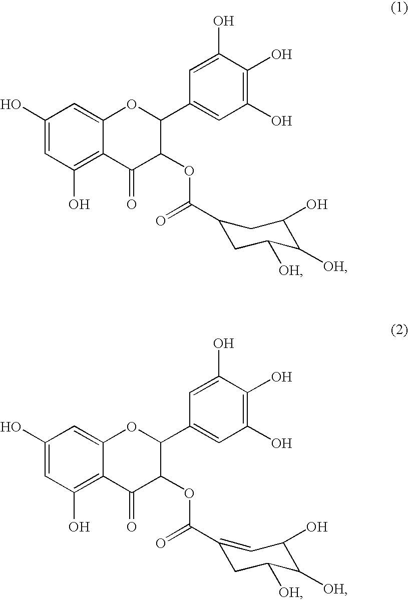

and pharmaceutically acceptable salts thereof.

Another aspect of the invention relates to a method of treating or preventing a renin-mediated condition in a subject in need thereof comprising administering to the subject a therapeutically effective amount of a compound of formula Ic:

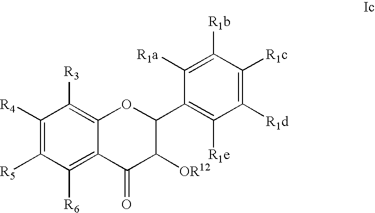

or a pharmaceutically acceptable salt thereof,
wherein, independently for each occurrence:

R₁ₐ, R_{1b}, R_{1c}, R_{1d}, R₁ₑ represent H, hydroxy, alkoxy, aralkyloxy, or aryloxy;

R₃, R₄, R₅, and R₆ represent H, hydroxy, alkoxy, aryloxy, or aralkyloxy; and

R₁₂ represents H, hydroxy, alkoxy, aralkyloxy, or aryloxy;

wherein any of the aforementioned alkoxy, aryloxy, aralkyloxy may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

In some embodiments, wherein R₁ₐ, R_{1b}, R_{1c}, R_{1d}, and R₁ₑ represent H, hydroxy, alkoxy, aralkyloxy, or aryloxy; provided that at least two of R₁ₐ, R_{1b}, R_{1c}, R_{1d}, and R₁ₑ are hydroxy;

R₃, R₄, R₅, and R₆ represent H, hydroxy, alkoxy, aryloxy, or aralkyloxy, provided that at least two of R₃, R₄, R₅, and R₆ are hydroxy; and

wherein any of the aforementioned alkoxy, aryloxy, aralkyloxy, cycloalkyl, or cycloalkenyl may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

In other embodiments, R₁ₐ, R_{1b}, R_{1c}, R_{1d}, and R₁ₑ represent H or hydroxy, and three of R₁ₐ, R_{1b}, R_{1c}, R_{1d}, and R₁ₑ are hydroxy.

In other embodiments, R₃, R₄, R₅, and R₆ represent H or hydroxy, and two of R₃, R₄, R₅, and R₆ are hydroxy.

In other embodiments, R₁₂ is OH.

Another aspect of the invention relates to a method of treating or preventing a renin-mediated condition in a subject in need thereof comprising administering to the subject a therapeutically effective amount of the following compound:

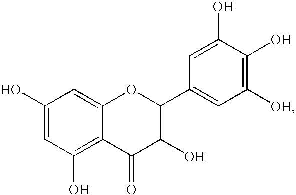

or a pharmaceutically acceptable salt thereof.

In some embodiments, the compound inhibits renin activity.

In other embodiments, the compound maintains electrolyte homeostasis.

In some embodiments, the cardiovascular condition selected from the group consisting of hypertension, severe hypertension, pulmonary hypertension (PH), malignant hypertension, isolated systolic hypertension, familial dyslipidemic hypertension, high blood pressure, atherosclerosis, unstable coronary syndrome, congestive heart failure, myocardial infarction, cardiac hypertrophy, cardiac fibrosis, cardiomyopathy postinfarction, unstable coronary syndrome, diastolic dysfunction, complications resulting from diabetes, diseases of the coronary vessels, elevated total cholesterol, low LDL cholesterol, peripheral vascular disease (PVD), peripheral artery disease (PAD), peripheral venous disorders, coronary arterial disease (CAD), restenosis following angioplasty, raised intra-ocular pressure, glaucoma, abnormal vascular growth, hyperaldosteronism, cerebrovascular diseases, metabolic disorder (Syndrome X), atrial fibrillation (AF), vascular inflammation, vasculitides or closure, aneurysm, angina, and restenosis of dialysis access grafts.

In some embodiments, the renal condition is selected from the group consisting of renal failure, chronic kidney disease, renoprotection, reduction of proteinuria, glomerulonephritis, nephrotic syndrome, renal fibrosis, acute interstitial nephritis (AlN), acute tubular nephritis (ATN), acute tubulo-interstitial nephritis, polycystic kidney disease (PKD), endothelial dysfunction, and microalbuminuria.

In some embodiments, the compound is administered in combination with one or more pharmaceutically acceptable carriers.

In some embodiments, the compound is administered in combination with at least one additional active agent selected from the group consisting of an angiotensin II receptor antagonist, an ACE inhibitor, a calcium channel blocker, an HMG-CO-A reductase inhibitor, an aldosterone synthase inhibitor, an aldosterone antagonist, an ACE/NEP inhibitor, a beta-blocker, an endothelin antagonist, and a diuretic.

In some embodiments, the subject is a mammal. In other embodiments, the subject is a primate, such as a human.

These embodiments of the present invention, other embodiments, and their features and characteristics, will be apparent from the description, drawings and claims that follow.

### BRIEF DESCRIPTION OF DRAWINGS

FIG. 1 depicts the inhibition of Renin by a compound of the present invention. The 50% inhibitory concentration is determined to be 10.2 μM based on the line-of-best-fit extrapolated from the inhibition curve (R²=0.94; n=30).

FIG. 2 depicts the interaction of a compound of the present invention with the Renin enzyme. Specifically, high binding affinity was recognized through multiple hydrogen bonds between the compound of the present invention and the amino acids Ser230, Tyr231, Ser84, and Thr85 present in the Renin binding site.

FIG. 3 depicts the mean reduction of Systolic blood pressure 24, 48, and 72 h after Tristenonol aglycone (Cmp 15 of the present invention) or Atenol® administration in rats with monocrotaline (n=10 rats per group).

FIG. 4 depicts the mean reduction of Diastolic blood pressure 24, 48, and 72 h after Tristenonol aglycone (Cmp 15 of the present invention) or Atenol® administration in rats with monocrotaline induced hypertension (n=10 rats per group).

FIG. 5 depicts the mean reduction of heart beats per minute after 24, 48, and 72 h of Tristenonol aglycone (Cmp 15 of the present invention) or Atenol® administration in rats with monocrotaline induced hypertension (n=10 rats per group).

### DETAILED DESCRIPTION OF THE INVENTION

For convenience, before further description of the disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The term “acyl” as used herein refers to the radical

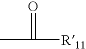

wherein R′₁₁ represents hydrogen, alkyl, alkenyl, alkynyl, or —(CH₂)ₘ—R₈₀, wherein R₈₀ is aryl, cycloalkyl, cycloalkenyl, heteroaryl or heterocyclyl; and m is an integer in the range 0 to 8, inclusive.

The term “alkyl” refers to a radical of a saturated straight or branched chain hydrocarbon group of, for example, 1-20 carbon atoms, or 1-12, 1-10, or 1-6 carbon atoms.

The term “alkenyl” refers to a radical of an unsaturated straight or branched chain hydrocarbon group of, for example, 2-20 carbon atoms, or 2-12, 2-10, or 2-6 carbon atoms, having at least one carbon-carbon double bond.

The term “alkynyl” refers to a radical of an unsaturated straight or branched chain hydrocarbon group of, for example, 2-20 carbon atoms, or 2-12, 2-10, or 2-6 carbon atoms, having at least one carbon-carbon triple bond.

The term “aliphatic” includes linear, branched, and cyclic alkanes, alkenes, or alkynes. In certain embodiments, aliphatic groups in the present invention are linear, branched or cyclic and have from 1 to about 20 carbon atoms.

The term “aralkyl” includes alkyl groups substituted with an aryl group or a heteroaryl group.

The term “heteroatom” includes an atom of any element other than carbon or hydrogen. Illustrative heteroatoms include boron, nitrogen, oxygen, phosphorus, sulfur and selenium, and alternatively oxygen, nitrogen or sulfur.

The term “halo” or “halogen” includes —F, —Cl, —Br, -or —I.

The term “perfluoro” refers to a hydrocarbon wherein all of the hydrogen atoms have been replaced with fluorine atoms. For example, —CF₃ is a perfluorinated methyl group.

The term “aryl” refers to a mono-, bi-, or other multi-carbocyclic, aromatic ring system. The aryl group can be fused optionally to one or more rings selected from aryls, cycloalkyls, and heterocyclyls. The aryl groups of this invention can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The term “heteroaryl” refers to a mono-, bi-, or multi-cyclic, aromatic ring system containing one, two, or three heteroatoms such as nitrogen, oxygen, and sulfur. Examples include pyrrole, furan, thiophene, imidazole, oxazole, thiazole, triazole, pyrazole, pyridine, pyrazine, pyridazine and pyrimidine, and the like. Heteroaryls can also be fused to non-aromatic rings.

The terms “heterocycle,” “heterocyclyl,” or “heterocyclic” refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkyl, alkenyl, alkynyl, aldehyde, alkylthio, alkanoyl, alkoxy, alkoxycarbonyl, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, oxo, nitro, sulfonate, sulfonyl, and thiol.

Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl. Heterocycles also include bridged bicyclic groups where a monocyclic heterocyclic group can be bridged by an alkylene group.

The heterocyclic or heteroaryl ring may be can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The terms “polycyclyl” and “polycyclic group” include structures with two or more rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms, e.g., three or more atoms are common to both rings, are termed “bridged” rings. Each of the rings of the polycycle may be substituted with such substituents as described above can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The term “carbocycle” includes an aromatic or non-aromatic ring in which each atom of the ring is carbon.

The terms “amine” and “amino” include both unsubstituted and substituted amines, e.g., a moiety that may be represented by the general formulas:

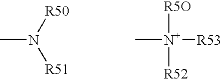

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

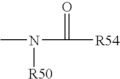

wherein R50 is as defined above, and R54 represents a hydrogen, an alkyl, an alkenyl or —(CH₂)ₘ—R61, where m and R61 are as defined above.

The term “amido” refers to an amino-substituted carbonyl and includes a moiety that may be represented by the general formula:

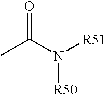

wherein R50 and R51 are as defined above. Certain embodiments of the amide in the present invention will not include imides that may be unstable.

The term “alkylthio” includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” includes such moieties as may be represented by the general formulas:

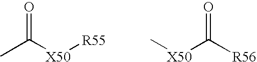

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” includes a moiety that may be represented by the general formula:

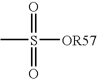

in which R57 is an electron pair, hydrogen, alkyl, cycloalkyl, or aryl.

The term “sulfate” includes a moiety that may be represented by the general formula:

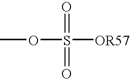

in which R57 is as defined above.

The term “sulfonamido” is art-recognized and includes a moiety that may be represented by the general formula:

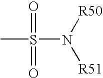

in which R50 and R51 are as defined above.

The term “sulfonyl” includes a moiety that may be represented by the general formula:

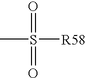

in which R58 is one of the following: hydrogen, alkyl, alkenyl, alkynyl, cycloalkyl, heterocyclyl, aryl or heteroaryl.

The term “sulfoxido” includes a moiety that may be represented by the general formula:

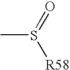

in which R58 is defined above.

The term “optionally substituted” or “substituted” is contemplated to include all permissible substituents of organic compounds. For example, substituted refers to a chemical group, such as alkyl, cycloalkyl, aryl, heteroaryl and the like, wherein one or more hydrogen atoms may be replaced with a substituent such as halogen, azide, alkyl, aralkyl, alkenyl, alklynyl, cycloalkyl, hydroxy, alkoxy, amino, amido, nitro, cyano, sulfhydryl, imino, phosphonate, phosphinate, carbonyl, carboxyl, silyl, ether, alkylthio, sulfonyl, sulfonamido, ketone, aldehyde, ester, heterocyclyl, aromatic or heteroaromatic moieties, perfluoroalkyl (e.g. —CF₃), acyl, and the like, or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)—. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The definition of each expression, e.g. alkyl, m, n, etc., when it occurs more than once in any structure, is intended to be independent of its definition elsewhere in the same structure unless otherwise indicated expressly or by the context.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

The term “hydrocarbon” includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991). The phrase “hydroxyl-protecting group” includes those groups intended to protect a hydroxyl group against undesirable reactions during synthetic procedures and includes, for example, benzyl or other suitable esters or ethers groups known in the art. The aforementioned protecting groups may be present in the compounds of the invention, and are not limited to use only during synthesis of the compounds of the invention. Thus, the presence of a protecting group is not intended to suggest that said group must be removed. For example, the compounds of the present invention may contain an ether group, such as a methoxymethyl ether, which is a known hydroxyl protecting group.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “effective amount” as used herein refers to the amount necessary to elicit the desired biological response. As will be appreciated by those of ordinary skill in this art, the effective amount of a drug may vary depending on such factors as the desired biological endpoint, the drug to be delivered, the composition of any additional active or inactive ingredients, the target tissue, etc.

The term “preventing”, when used in relation to a condition, such as a rennin mediated condition, cardiovascular condition or renal condition, or any other medical disease or condition, is well understood in the art, and includes administration of a composition which reduces the frequency of, or delays the onset of, symptoms of a medical condition in a subject relative to a subject which does not receive the composition.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “synergistic” is art recognized and refers to two or more components working together so that the total effect is greater than the sum of the components.

The term “treating” is art-recognized and refers to curing as well as ameliorating at least one symptom of any condition or disorder.

As used herein, the term “inhibitor” refers to a molecule that binds to an enzyme and decreases the enzymes activity activity. The binding of an inhibitor can stop a substrate from entering the enzyme's active site and/or hinder the enzyme from catalyzing its reaction Inhibitor binding is either reversible or irreversible. Irreversible inhibitors usually react with the enzyme and change it chemically. These inhibitors modify key acid residues needed for enzymatic activity. In contrast, reversible inhibitors bind non-covalently and different types of inhibition are produced depending on whether these inhibitors bind the enzyme, the enzyme-substrate complex, or both.

As used herein, the term “substrate” refers to a molecule upon which an enzyme acts. Enzymes catalyze chemical reactions involving the substrate(s). The substrate binds with the enzyme's active site, and an enzyme-substrate complex is formed. The substrate is broken down into a product and is released from the active site. The active site is now free to accept another substrate molecule.

As used herein, the term “Renin” refers to a circulating enzyme that participates in the body's renin-angiotensin system (RAS) that mediates extracellular volume (i.e. that of your blood, lymph and other body fluids), and arterial vasoconstriction (i.e. the tone of the musculature of arteries). Renin activates the renin-angiotensin system by cleaving angiotensinogen produced by the liver to yield angiotensin I, which is further converted to angiotensin II by angiotensin converting enzyme II (ACE2). Angiotensin II then constricts blood vessels, increases secretion of ADH and aldosterone, and stimulates the hypothalamus to activate the thirst reflex, each of which leads to an increase in blood pressure.

As used herein, the term “angiotensin” refers to an oligopeptide in the blood that causes vasoconstriction, increased blood pressure, and release of aldosterone from the adrenal cortex. It is a hormone and is derived from the precursor molecule angiotensinogen, a serum globulin produced in the liver. It plays an important role in the renin-angiotensin system.

A “patient,” “subject” or “host” to be treated by the subject method includes either a human or non-human animal.

The compounds of the present invention may be used in the form of pharmaceutically acceptable salts derived from inorganic or organic acids. The term “pharmaceutically acceptable salt” includes those salts that are, within the scope of sound medical judgment, suitable for use in contact with the tissues of humans and lower animals without undue toxicity, irritation, and allergic response, and are commensurate with a reasonable benefit/risk ratio. Pharmaceutically acceptable salts are well known in the art. For example, S. M. Berge, et al. (1997) describe pharmaceutically-acceptable salts in J. Pharm. Sci., 66:1-19. The salts may be prepared in situ during the final isolation and purification of the compounds of the invention or separately by reacting a free base function with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates, such as dimethyl, diethyl, dibutyl and diamyl sulfates; long-chain halides such as decyl, lauryl, myristyl and stearyl chlorides, bromides and iodides; or arylalkyl halides, such as benzyl and phenethyl bromides and others. Water- or oil-soluble or -dispersible products are thereby obtained.

Examples of acids that may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, and citric acid.

The present invention includes all salts and all crystalline forms of such salts. Basic addition salts can be prepared in situ during the final isolation and purification of compounds of this invention by combining a carboxylic acid-containing group with a suitable base such as the hydroxide, carbonate, or bicarbonate of a pharmaceutically acceptable metal cation or with ammonia or an organic primary, secondary, or tertiary amine. Pharmaceutically acceptable basic addition salts include cations based on alkali metals or alkaline earth metals such as lithium, sodium, potassium, calcium, magnesium, and aluminum salts, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

### Compounds

Isolated compounds have been identified from extracts showing inhibitory activity against the Renin enzyme. Compounds of the present invention have also been synthesized (>98% purity) and show Renin inhibition activity. Compounds of the present invention include flavononols, such as Tristenonol (5,7-dihydroxy-4-oxo-2-(3,4,5-trihydroxyphenyl)chroman-3-yl-3,4,5-trihydroxy-cyclohexanecarboxylate).

The pure and isolated flavononol compounds of the present invention are represented by formula I:

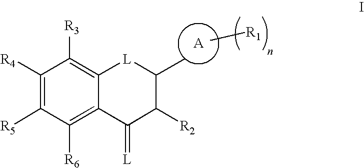

wherein, independently for each occurrence:

R₁ represents alkoxy, alkenyloxy, alkynyloxy, aryloxy, arylalkyloxy, hydroxy, —OC(O)—R₇, alkyl, alkenyl, alkynyl, acetyl, formyl, halide, cyano, nitro, SH, amino, amido, sulfonyl, or sulfonamido;

R₂ represents OH or

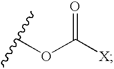

R₃, R₄, R₅, and R₆ represent H, hydroxy, alkoxy, alkenyloxy, alkynyloxy, aryloxy, aralkyloxy; —OC(O)—R₇, alkyl, alkenyl, alkynyl, aralkyl, acetyl, formyl, halide, cyano, nitro, SH, amino, amido, sulfonyl, or sulfonamido;

R₇ represents H, alkyl, alkenyl, alkynyl, cycloalkyl, aryl, arylalkyl or a carbohydrate;

A represents an aryl group;

L represents O, S, or NR;

R represents H, hydroxy, alkyl, alkenyl, alkynyl, aralkyl, acetyl, formyl, or sulfonyl;

X represents a carbohydrate, a cycloalkyl, or a cycloalkenyl group; and

n represents an integer from 1 to 5, inclusive;

wherein any of the aforementioned alkoxy, alkenyloxy, alkynyloxy, aryloxy, aralkyloxy, alkyl, alkenyl, alkynyl, cycloalkyl, cycloalkenyl, aryl and aralkyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

In another embodiment, the esterified flavonolol compounds of the present invention are represented by formula I, wherein, independently for each occurrence:

R₁ represents H, alkoxy, aryloxy, aralkyloxy, hydroxy, —OC(O)—R₇, alkyl, acetyl, formyl, or halide;

R₂ represents

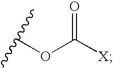

R₃, R₄, R₅, and R₆ represent H, alkoxy, aryloxy, aralkyloxy; —OC(O)—R₇, alkyl, aralkyl, acetyl, formyl, or halide;

R₇ represents H, alkyl, aryl, or arylalkyl;

A represents an aryl group;

L represents O;

X represents a carbohydrate, a cycloalkyl, or a cycloalkenyl group; and

n represents an integer from 1 to 5, inclusive;

wherein any of the aforementioned alkoxy, alkenyloxy, alkynyloxy, aryloxy, aralkyloxy, alkyl, alkenyl, alkynyl, aryl, aralkyl, cycloalkyl and cycloalkenyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

The carbohydrate may be a monosaccharide such as arabinose, lyxose, ribose, rhamnose, deoxyribose, xylose, ribulose, xylulose, allose, altrose, galactose, glucose, gulose, idose, mannose, talose, fructose, mannose, psicose, sorbose, or tagatose. In another embodiment, the carbohydrate may be a disaccharide such as sucrose, lactose, maltose, trehalose or cellobiose. In another embodiment, the carbohydrate may be an oligosaccharide such as raffinose, maltodextrin, and cyclodextrin. In another embodiment, the carbohydrate may be a polysaccharide such as starch, glycogen, dextran, and cellulose.

In another embodiment, R₂ is OH.

In another embodiment, the flavononol compounds are represented by formula I, wherein L is O.

In another embodiment, the flavononol compounds are represented by formula I, wherein R₃, R₄, R₅ and R₆ are each independently H or hydroxy, wherein at least two of R₃, R₄, R₅ and R₆ are hydroxy.

In another embodiment, the flavononol compounds are represented by formula I. wherein R₁ is hydroxy, and n is equal to 2 or 3.

In another embodiment, the flavononol compounds are represented by formula I, wherein A is a benzene ring.

In another embodiment, the flavononol compounds are represented by formula I, wherein X is a carbohydrate.

In another embodiment, the flavononol compounds are represented by formula I, wherein X is a cycloalkyl or cycloalkenyl group; and wherein the cycloalkyl or cycloalkenyl group is substituted with 1 to 3 hydroxy groups.

In another embodiment, the flavononol compounds of the present invention are represented by formula Ia:

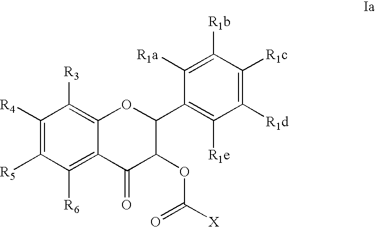

wherein, independently for each occurrence:

R₁ₐ, R_{1b}, R_{1c}, R_{1d}, R₁ₑ represent H, hydroxy, alkoxy, aralkyloxy, or aryloxy;

R₃, R₄, R₅, and R₆ represent H, hydroxy, alkoxy, aryloxy, or aralkyloxy; and

X represents a carbohydrate, a cycloalkyl, or a cycloalkenyl group;

wherein any of the aforementioned alkoxy, aryloxy, aralkyloxy may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

In another embodiment, the flavononols of the present invention are represented by formula Ia, wherein independently for each occurrence:

R₁ₐ, R_{1b}, R_{1c}, R_{1d}, and R₁ₑ represent H, hydroxy, alkoxy, aralkyloxy, or aryloxy; provided that at least two of R₁ₐ, R_{1b}, R_{1c}, R_{1d}, and R₁ₑ are hydroxy;

R₃, R₄, R₅, and R₆ represent H, hydroxy, alkoxy, aryloxy, or aralkyloxy, provided that at least two of R₃, R₄, R₅, and R₆ are hydroxy; and

X is carbohydrate, cycloalkyl, or cycloalkenyl;

wherein any of the aforementioned alkoxy, aryloxy, aralkyloxy, cycloalkyl, or cycloalkenyl may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

In another embodiment, the flavononol compounds of the present invention are represented by formula Ia, wherein: R₁ₐ, R_{1b}, R_{1c}, R_{1d}, and R₁ₑ represent H or hydroxy, and three of R₁ₐ, R_{1b}, R_{1c}, R_{1d}, and R₁ₑ are hydroxy.

In another embodiment, the flavononol compounds of the present invention are represented by formula Ia, wherein: R₃, R₄, R₅, and R₆ represent H or hydroxy, and two of R₃, R₄, R₅, and R₆ are hydroxy.

In another embodiment, the flavononol compounds of the present invention are represented by formula Ia, wherein: X is a carbohydrate selected from the group consisting of a monosaccharide, a disaccharide, an oligosaccharide, and a polysaccharide.

In another embodiment, X is a carbohydrate selected from the group consisting of arabinose, lyxose, ribose, rhamnose, deoxyribose, xylose, ribulose, xylulose, allose, altrose, galactose, glucose, gulose, idose, mannose, talose, fructose, psicose, sorbose, tagatose, sucrose, lactose, maltose, trehalose or cellobiose, raffinose, maltodextrin, cyclodextrin, starch, glycogen, dextran, and cellulose.

In yet another embodiment, X is rhamnose.

In another embodiment, X is a cycloalkyl or cyloalkynyl group, wherein the cycloalkyl or cycloalkenyl group may be substituted with one to three hydroxy groups.

In another embodiment, the flavononol compounds of the present invention are represented by formula Ib,

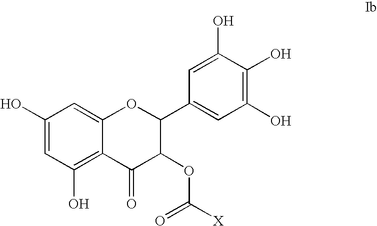

wherein X represents a carbohydrate, a cycloalkyl, or a cycloalkenyl, wherein the cycloalkyl or cycloalkenyl may be substituted with one to three hydroxy groups. The carbohydrate may be a monosaccharide such as arabinose, lyxose, ribose, rhamnose, deoxyribose, xylose, ribulose, xylulose, allose, altrose, galactose, glucose, gulose, idose, mannose, talose, fructose, psicose, sorbose, or tagatose. In another embodiment, the carbohydrate may be a disaccharide such as sucrose, lactose, maltose, trehalose or cellobiose. In another embodiment, the carbohydrate may be an oligosaccharide such as raffinose, maltodextrin, and cyclodextrin. In another embodiment, the carbohydrate may be a polysaccharide such as starch, glycogen, dextran, and cellulose.

Esterification on the 3′-O of Ring C on the flavononol proceeds through reaction of the acid form of the above listed carbohydrate under standard esterification conditions.

In another embodiment, X is a cyclohexyl or cyclohexenyl. In another embodiment, X is:

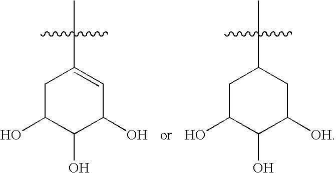

In a further embodiment, the flavononol of the present invention is:

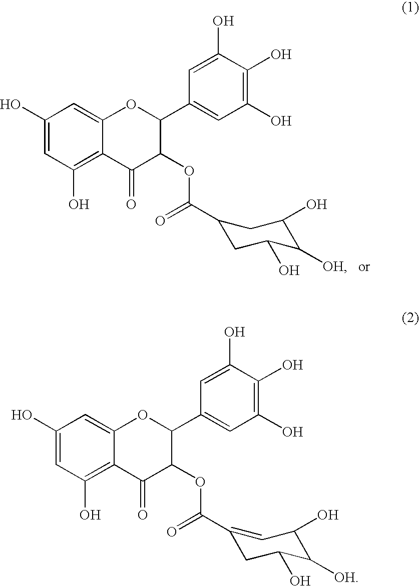

The aforementioned compounds may be pure and isolated, e.g., by chemical synthesis and/or extraction from a botanical, or the compounds may be present in a mixture. In some embodiments, the aforementioned compounds are present in an amount of about 5 to 90% in a mixture, such as a mixture obtained by extraction of a botanical. In other embodiments, the aforementioned compounds may be present in an amount of about 5, 10, 15, 20, 25, 30, 35, 40, 45, 50, 55, 60, 65, 70, 75, 80, 85, 90 or 95% in a mixture.

In another embodiment, the compound is:

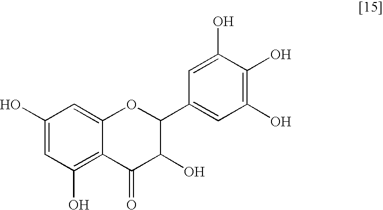

### Synthesis of Compounds of the Present Invention

The compounds obtained from an extract may be further purified and/or modified by synthetic organic methods well-known in the art.

The compounds of the invention may also be obtained by synthetic organic method well known in the art. For example, Scheme I depicts a general route to the synthesis of flavononols. The starting material is an R_{b}-substituted acetyl phenone (i) and benzaldehyde, where R_{b}-groups are alkoxy, alkenyloxy, alkynyloxy, aryloxy, arylalkyloxy, hydroxy, —OC(O)—R₇, alkyl, alkenyl, alkynyl, acetyl, formyl, halide, cyano, nitro, SH, amino, amido, sulfonyl, or sulfonamido. The R_{b}-groups may additionally be one the aforementioned groups protected with a suitable protecting group to prevent undesired side reactions. For example, OH may be protected by protecting groups such as methoxymethyl (MOM), or NH₂ may be protected with CBZ, etc. The starting material (i) undergoes a base-catalyzed aldol condensation or acid-mediated adolization with the substituted benzaldehyde to yield a chalcone (ii). (See March 1994, Streitweiser 1992). The chalcone is then expoxidized to form epoxy chalcone (iii) or subjected to based-catalyzed cyclization to form flavonone (iv). (See March 1994, Carey and Sundberg 1992). The epoxy chalcone is subjected to either acid, free radical or Lewis acid-catalyzed cyclization to yield flavononol (v). (See March 1994, Carey and Sundberg 1992). Flavonone (iv) undergoes an oxidation reaction to yield the flavononol (See March 1994, Carey and Sundberg 1992).

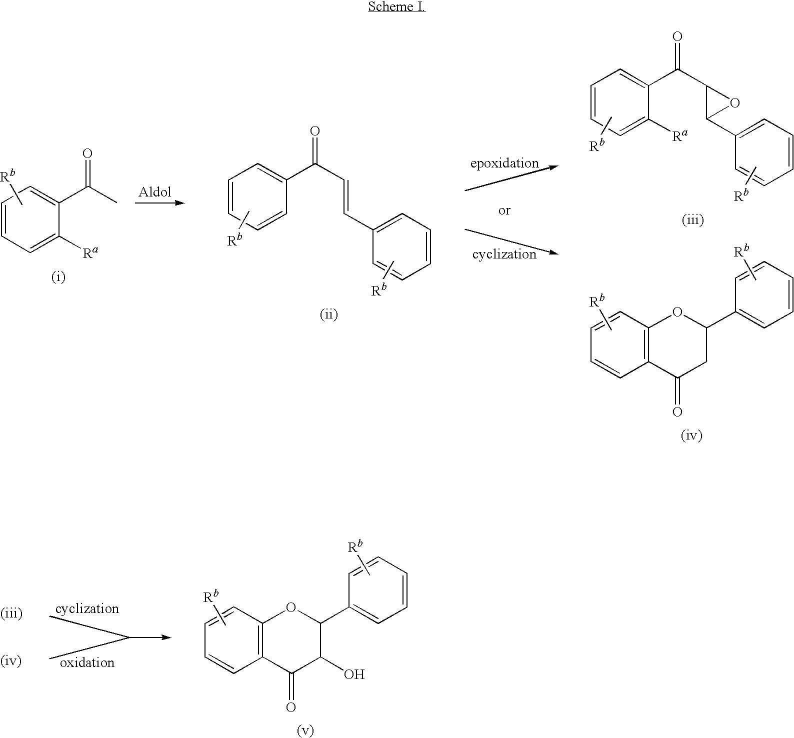

The flavononol (v) as described in Scheme I is esterified under acid catalysis with a carboxylic acid, for example, 3,4,5-trihydroxy cyclohexane carboxylic acid (e.g. shikimic acid) or glycosylated on the 3-OH group of the C ring to yield esterified flavononol (vii). (See March 1994, Streitweiser 1992). Additionally, the flavononol can be reduced at the C-2 carbonyl to yield a leucoanthocyanidin (vii). (See March 1994, Carey and Sundberg 1992). The flavononol and leucoanthocyanidin compounds can be further separated and purified so as to obtain pure and isolated anthocyanadins by methods known in the art, such as flash column chromatography, HPLC, recrystallization, etc.

Scheme II represents a synthetic method used to obtain a specific flavononol, the Tristenonol aglycone. The Tristenonol aglycone was synthesized in five steps by coupling methoxymethyl (MOM) protected acetophenone and benzaldehyde, 10 and 12 respectively. The chalcone formed through this reaction was epoxidized using hydrogen peroxide to give compound 14, and the compound 14 was cyclized with the aryl OH (from MOM deprotection during the same reaction) to give the Tristenonol aglycone [15; 5,7-dihydroxy-4-oxo-2-(3,4,5-trihydroxyphenyl)chroman-3-yl-3,4,5-trihydroxycyclohexanecarboxylate] in 66% overall yield.

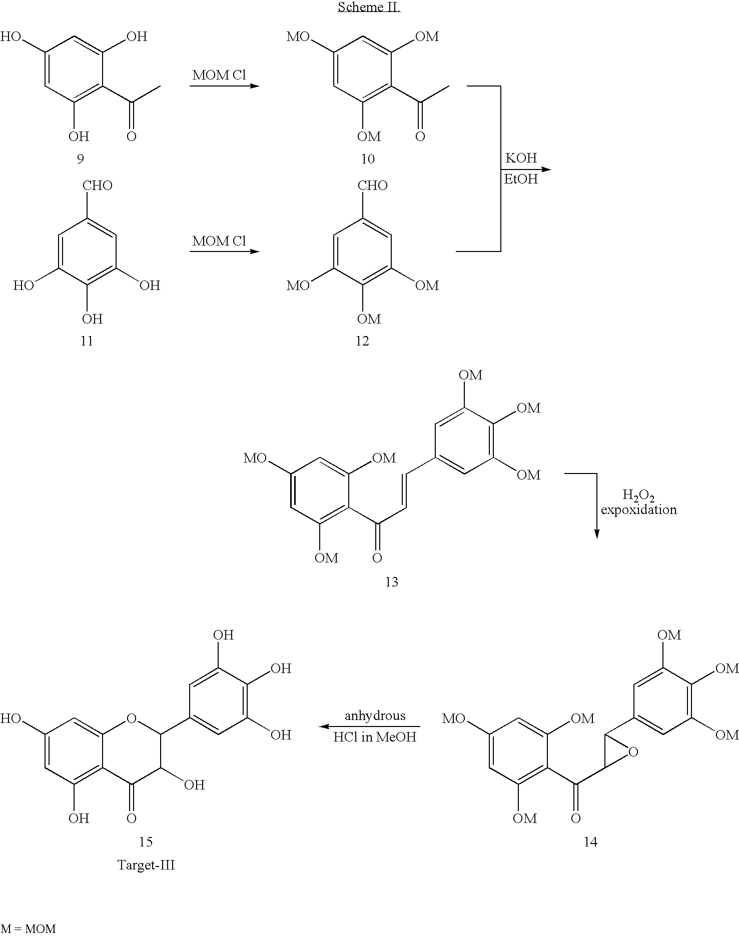

The esterified flavononols of the present invention may be prepared from flavononol (v) of Scheme I according to Scheme III:

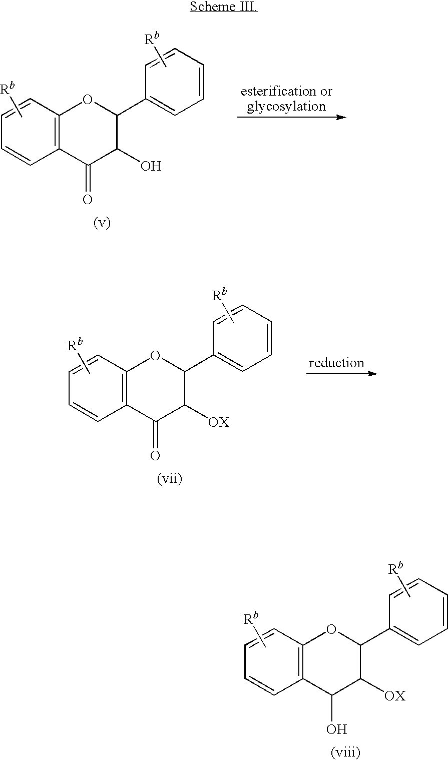

### Methods of Treatment

The present invention also relates in part to a method of treating or preventing a cardiovascular diseases, as well as maintaining electrolyte homeostasis and proper kidney function in a subject comprising administering to a subject in need thereof a therapeutically effective amount of a compound or composition of the present invention. The present invention also relates to a method of treating or preventing a Renin mediated condition.

In some embodiments, the present invention relates to a method of treating or preventing a cardiovascular or renal condition comprising administering to a subject in need thereof a therapeutically effective amount of any of the aforementioned compounds. In some embodiments, the compound inhibits renin activity. In other embodiments, the compound maintains electrolyte homeostasis.

In some embodiments, the cardiovascular condition selected from the group consisting of hypertension, severe hypertension, pulmonary hypertension (PH), malignant hypertension, isolated systolic hypertension, familial dyslipidemic hypertension, high blood pressure, atherosclerosis, unstable coronary syndrome, congestive heart failure, myocardial infarction, cardiac hypertrophy, cardiac fibrosis, cardiomyopathy postinfarction, unstable coronary syndrome, diastolic dysfunction, complications resulting from diabetes, diseases of the coronary vessels, elevated total cholesterol, low LDL cholesterol, peripheral vascular disease (PVD), peripheral artery disease (PAD), peripheral venous disorders, coronary arterial disease (CAD), restenosis following angioplasty, raised intra-ocular pressure, glaucoma, abnormal vascular growth, hyperaldosteronism, cerebrovascular diseases, metabolic disorder (Syndrome X), atrial fibrillation (AF), vascular inflammation, vasculitides or closure, aneurysm, angina, and restenosis of dialysis access grafts.

In some embodiments, the cardiovascular condition in high blood pressure.

In some embodiments, the renal condition is selected from the group consisting of renal failure, chronic kidney disease, renoprotection, reduction of proteinuria, glomerulonephritis, nephrotic syndrome, renal fibrosis, acute interstitial nephritis (AIN), acute tubular nephritis (ATN), acute tubulo-interstitial nephritis, polycystic kidney disease (PKD), endothelial dysfunction, and microalbuminuria.

In some embodiments, the compound is administered in combination with one or more pharmaceutically acceptable carriers.

In some embodiments, the compound is administered in combination with at least one additional active agent selected from the group consisting of an angiotensin II receptor antagonist, an ACE inhibitor, a calcium channel blocker, an HMG-CO-A reductase inhibitor, an aldosterone synthase inhibitor, an aldosterone antagonist, an ACE/NEP inhibitor, a beta-blocker, an endothelin antagonist, and a diuretic.

The compounds that may be used in the methods of the present invention are described below. The compounds are understood to include the listed compound and pharmaceutically acceptable salts thereof.

Angiotensin converting enzyme inhibitors (ACE inhibitor) refers to a compound having the ability to block, partially or completely, the rapid enzymatic conversion of the physiologically inactive decapeptide form of angiotensin “Angiotensin I) to the vasoconstrictive octapeptide form of angiotensin (Angiotensin II). Examples of ACE inhibitors suitable for use herein are for instance the following compounds: AB-103, ancovenin, benazeprilat, BRL-36378, BW-A575C, CGS-13928C, CL242817, CV-5975, Equaten, EU4865, EU-4867, EU-5476, foroxymithine, FPL 66564, FR-900456, Hoe-065, 15B2, indolapril, ketomethylureas, KRI-1177, KRI-1230, L681176, libenzapril, MCD, MDL-27088, MDL-27467A, moveltipril, MS-41, nicotianamine, pentopril, phenacein, pivopril, rentiapril, RG-5975, RG-6134, RG-6207, RGH0399, ROO-911, RS-10085-197, RS-2039, RS 5139, RS 86127, RU-44403, S-8308, SA-291, spiraprilat, SQ26900, SQ-28084, SQ-28370, SQ-28940, SQ-31440, Synecor, utibapril, WF-10129, Wy-44221, Wy-44655, Y-23785, Yissum, P-0154, zabicipril, Asahi Brewery AB-47, alatriopril, BMS 182657, Asahi Chemical C-111, Asahi Chemical C-112, Dainippon DU-1777, mixanpril, Prentyl, zofenoprilat, 1 (1-carboxy-6-(4-piperidinyl)hexyl)amino)-1-oxopropyl octahydro-1H-indole-2-carboxylic acid, Bioproject BP1.137, Chiesi CHF 1514, Fisons FPL-66564, idrapril, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, trandolapril, trandolaprilat, ceranapril, moexipril, quinaprilat, and spirapril.

Examples of angiotensin converting enzyme/neutral endopeptidase (ACE/NEP) inhibitors include sampatrilat, fasidotril, fasidotrilate omapatrilate, and enalaprilat.

Examples of angiotensin II antagonists include aralasin acetate, candesartan cilexetil, CGP-63170, EMD-66397, KT3671, LR-B/081, valsartan, A-81282, BIBR-363, BIBS-222, BMS-184698, candesartan, CV-11194, EXP-3174, KW-3433, L-161177, L-162154, LR-B/057, LY-235656, PD-150304, U-96849, U-97018, UP-275-22, WAY-126227, WK-1492.2K, YM-31472, losaprtan potassium, E-4177, EMD-73495, eprosartan, HN-65021, irbesartan, L-159282, ME-3221, SL-91.0102, Tasosartan, Telmisartan, UP-269-6, YM-358, CGP49870, GA-0056, L-159689, L-162234, L-162441, L-163007, PD-123177, A-81988, BMS-180560, CGP-38560A, CGP48369, DA-2079, DE-3489, DuP-167, EXP-063, EXP-6155, EXP-6803, EXP-7711, EXP-9270, FK-739, HR-720, ICI-D6888, ICI-D7155, ICI-D8731, isoteoline, KRI-1177, L-158809, L-158978, L-159874, LR B087, LY-285434, LY-302289, LY-315995, RG-13647, RWJ-38970, RWJ46458, S-8307, S-8308, saprisartan, saralasin, Sarmesin, WK-1360, X-6803, ZD-6888, ZD-7155, ZD-8731, BIBS39, CI-996, DMP-811, DuP-532, EXP-929, L-163017, LY-301875, XH-148, XR-510, zolasartan, and PD-123319.

Calcium channel blockers (CCBs) include dihydropyridines (DHPs) and non-DHPs, such as diltiazem-type and verapamil-type CCBs. For example, calcium channel blockers include amlodipine, felodipine, ryosidine, isradipine, lacidipine, nicardipine, nifedipine, niguldipine, niludipine, nimodipine, nisoldipine, nitrendipine and nivaldipine, and non-DHP calcium channel blockers include flunarizine, prenylamine, diltiazem, fendiline, gallopamil, mibefradil, anipamil, tiapamil, and verapamil.

Hydroxy-3-methyl-glutaryl-CoA reductase (HMG-Co-A reductase) inhibitors include the class of drugs known as statins, such as atorvastatin, cerivastatin, compactin, dalvastatin, dihydrocompactin, fluindostatin, fluvastatin, lovastatin, pitavastatin, mevastatin, pravastatin, rivastatin, simvastatin, rosuvastatin, and velostatin.

Aldosterone synthase inhibitors/aldosterone antagonists include eplerenone, (+)-fadrozole, spironolactone, anastrozole, exemesartane, and canrenone.

Beta-blockers suitable for use in the present invention acebutolol, atenolol, betaxolol, bisoprolol, carteolol, carvedilol, esmolol, labetalol, metoprolol, nadolol, oxprenolol, penbutolol, pindolol, propranolol, sotalol, and timolol.

Endothelin antagonists include bosentan, enrasentan, atrasentan, darusentan, BMS 193884, sitaxentan, YM 598, S 0139, J 104132, and tezosentan.

Diuretics, such as bumetanide, ethacrynic acid, furosemide, torsemide, hydrochlorothiazide, indapamide, metazolone, amiloride, hydroflumethoazide, methylchlothiazide, metolazone, dichlorphenamide, triamterene, chlorothialidone, and chlorothiazide.

In another embodiment, the invention relates to a method of inhibiting renin in a subject in thereof, comprising administering to the subject an effective amount of any of the aforementioned compounds. In other embodiments, the invention relates to a method of maintaining electrolyte homeostasis in a subject in need thereof, comprising administering to the subject an effective amount of any of the aforementioned compounds.

In some embodiments, the subject is a mammal. In other embodiments, the subject is a primate, such as a human.

### Pharmaceutical and Personal Healthcare Formulations

The antiinfective compositions of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compositions of the present invention may be formulated as eye drops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In the aforementioned formulations, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

### EXEMPLIFICATION

### Methods

A. Renin Inhibition

Compounds of the present invention were dissolved in pure DMSO. Serial dilutions were generated so that the concentration of DMSO was no more than 1% (v/v) in experimental wells. The samples were incubated with a renin substrate which is linked to a fluorophore (EDANS) and a fluorescence quenching molecule (Dabcyl) and either a Human Renin enzyme solution or assay buffer. The non-enzyme containing wells served as background wells. The product of the enzymatic reactions possesses enhanced fluorescence emission at 495 nm when excited at 340 nm. Fluorescence emission was monitored at 495 nm (Ex 340 nm) to determine the change in enzyme activity in the presence of composition of the present invention compared to positive (enzyme only) and negative (no enzyme) controls.

B. DART Time-Of-Flight Mass Spectrometry

The JEOL DART™ AccuTOF mass spectrometer (JMS-T 100LC; Jeol USA, Peabody, Mass.) used for chemical analysis requires no sample preparation and yields masses with accuracies to 0.0001 mass units (R. B. Cody, J. A Laramée, J. M. Nilles and H. D. Durst, 2005. Direct Analysis in Real Time (DART™) Mass Spectrometry. JEOL News 40:8-12). For positive ion mode (DART+), the needle voltage was set to 3500V, heating element to 300° C., electrode 1 to 150V, electrode 2 to 250V, and helium gas flow to 2.52 liters per minute. For the mass spectrometer, the following settings were loaded: orifice 1 set to 10V, ring lens voltage set to 5V, and orifice 2 set to 5V. The peak voltage was set to 1000V in order to give peak resolution beginning at 100 m/z. The microchannel plate detector (MCP) voltage was set at 2600V. Calibrations were performed internally with each sample using a 10% (w/v) solution of PEG that provided mass markers throughout the required mass range 100-1000 m/z. Calibration tolerances were held to 5 mmu.

C. Determination of Chemical Structures

Molecular formula and chemical structure was identified and confirmed by elemental composition and isotope matching programs in the Jeol MassCenterMain Suite software (MassCenter Main, Version 1.3.0.0; JEOL USA Inc.: Peabody, Mass., USA, Copyright® 2001-2004). In addition, molecular formulas and structure identifications were searched against the NIST/NIH/EPA Mass Spec Database (S. Stein, Y. Mirokhin, D. Tchekhovskoi, G. Mallard, A. Mikaia, V. Zaikin, J. Little, D. Zhu, C. Clifton, and D. Sparkman, 2005. The NIST mass spectral search program for the NIST/EPA/NIH mass spectral library—Version 2.0d. National Institute of Standards and Technology, Gaithersburg, Md.), the Dictionary of Natural Products (Chapman & Hall: Dictionary of Natural Products on DVD—Version 16:2. CRC Press, Boca Raton, Fla., 2008), and the Chemical Abstract Services structure search (chembiofinder.cambridgesoft.com).

D. Pharmacokinetic Analysis

Pharmacokinetic analysis on a mixture of chemicals containing compounds of the present invention was conducted in two parts. In the first part, six healthy consenting adults ranging in age from 23 to 50 were hospitalized 24 h prior to the initiation of the study and provided a diet free of flavonoids. A certified individual collected blood samples at several time intervals between 0 and 480 min after ingestion of a 175-mg dose of an extract containing compounds of the present invention in lozenge form. Blood samples were handled with approved protocols and precautions, centrifuged to remove cells and the serum fraction was collected and frozen. Blood was not treated with heparin to avoid any analytical interference.

In the second part of the study, a single healthy adult male (age 50) was recruited. A certified individual collected blood samples over the same time course as described above. Blood samples were handled with approved protocols and precautions. The subject fasted for 24 h prior to the initiation of blood collection, and received only water and foods absent in flavonoids during the course of the study. Blood samples were taken at several time intervals between 0 and 360 min. A mixture of chemicals containing compounds of the present invention (350 mg) was dissolved in 8 oz of water and administered immediately after the time-zero blood sample was collected. Blood samples were collected and processed as described above for analysis.

The cells were removed from the blood samples by centrifugation and the serum was collected. Serum samples were prepared for DART TOF-MS analysis by extraction with an equal volume of neat ethanol (USP) to minimize background of proteins, peptides, and polysaccharides present in serum. The ethanol extract was centrifuged for 10 min at 4° C., the supernatant was removed, concentrated to 200 μL volume, and 50 μL of an internal standard was added.

E. Molecular Modeling and ADMET Predictions

Molecular modeling software (Accelrys Discovery Studio 2.5) was used to predict the Absorption, Distribution, Metabolism, Excretion, and Toxicity (ADMET) properties of the pharmaceutical compositions of the present invention. The physicochemical properties of the compounds of the present invention were used for the ADMET evaluations.

Renin (PDB file: 3GW5) was downloaded from the RCSB protein databank (http://www.rcsb.org/pdb/home/home.do) and was used for molecular modeling with CDOCKER algorithms contained in Accelrys Discovery Studio 2.5.

E. Animal Studies

Animal Care and Handeling. Sprague-Dawley rats (Charles River Laboratories, Wilmington, Mass.) 60 days old and weighing 140-160 g were utilized. Animals were kept in standard autoclaved rodent cages with ad libitum food (Harlan Teklad Irradiated Rodent Diet) and autoclaved water. The rats were housed on Harlan Tek-chip pelleted paper in static micro isolators maintained at 22° C., 60% relative humidity, and under a 12 h light cycle. The animals were kept at the facility for 5 days to get accustomed to the environment after the arrival from the vendor. All animal care procedures complied with the recommendations of the Guide for Care and Use of Laboratory Animals with respect to restraint, husbandry, surgical procedures, feed and fluid regulation, and veterinary care.
Induction of hypertension and the treatment. Rats were injected subcutaneously with monocrotaline (MCT, 60 mg kg⁻¹) and monitored for 12 days for the development of pulmonary inflammation and initiation of the pulmonary hypertensive response. After the incubation period, Tristenonol aglycone (Cmp. 15 of the present invention) was administrated orally to the animals in three different doses (120, 90, and 60 mg kg⁻¹ body weights). Blood pressure (BP; Systolic and Diastolic) and heart beats were recorded three times a day 24, 48, and 72 h after administration of compound BP. The study design and treatments are summarized in Table 1.

TABLE 1
Animal treatment regimes including control,
experimental (Tristenonol aglycone [15] of the
present invention) and a positive control (Atenol ®).

| | | Dose (mg kg⁻¹) | | |
|---|---|---|---|---|
| Group | Compound | Body Wt. | | No. Animals |
| 1 | Control (no monocrotaline) | 10 | | 10 |
| 2 | Untreated control (plus | 10 | | 10 |
| | monocrotaline) | | | |
| 3 | Tristenonol [15] High dose | 120 | mg kg⁻¹ | 10 |
| 4 | Tristenonol [15] Medium dose | 90 | mg kg⁻¹ | 10 |
| 5 | Tristenonol [15] Low dose | 60 | mg kg⁻¹ | 10 |
| 6 | Atenol ® Control | 120 | mg kg⁻¹ | 10 |

All surviving animals were euthanized on day 28 by carbon dioxide inhalation or were euthanized earlier if they became moribund. The animals were observed and if they were found incapable of eating or drinking, or the temperature of the animals was too low to sustain viability, the animals were sacrificed.

Test Articles and Animal Groups. Three doses of Tristenonol aglycone (Cmp 15 of the present invention) were tested. The animals were randomized before and grouped in to 10 animals per group. For positive controls used the commercially available drug Atenol® was used.

Rats were sorted into six groups of ten and treated according to the protocol in Table 1. Group 1 rats did not receive monocrotaline injection (baseline control). All remaining groups were administered monocrotaline. Group 2 rats did not receive any additional treatment (untreated control). Groups 3, 4 and 5 received Tristenonol aglycone (Cmp 15 of the present invention) at 160, 90, and 60 mg respectively per kg body weight 12 days after induction of hypertension with monocrotaline. Positive Control Group 6 was given the commercial drug Atenol® at 120 mg kg⁻¹.

Systolic blood pressure, diastolic blood pressure and heart beats were recorded in the morning, afternoon and evening three times, 24, 48, and 72 h after Tristenonol aglycone (Cmp 15) and Atenol® administration.

Protocol for the Measurement of the Blood Pressure. A tail-cuff was placed on the tail to occlude the blood flow. Upon deflation a non-invasive blood pressure sensors, placed distal to the occlusion cuff, was utilized to monitor the blood pressure.
Volume Pressure Recording. The volume pressure recording sensor utilizes a specially designed differential pressure transducer to non-invasively measure the blood volume in the tail. Volume pressure recording actually measured six blood pressure parameters simultaneously: systolic blood pressure, diastolic blood pressure, mean blood pressure, heart pulse rate, tail blood volume and tail blood flow. Since volume pressure recording utilizes a volumetric method to measure the blood flow and blood volume in the tail, there were no measurement artifacts related to ambient light; movement artifact was also greatly reduced. In addition, volume pressure recording was not dependent on the skin pigmentation of the animal. Dark-skinned animals have no negative effect on volume pressure recording measurements. Special attention was afforded to the length of the occlusion cuff with volume pressure recording in order to derive the most accurate blood pressure readings.
Endpoint Measurements. Each animal was sacrificed when the animal reached the predetermined endpoint of being moribund or when the animals remained system free. The animals were observed and if found incapable of eating or drinking or if their body temperature were too low to sustain life, the animals were sacrificed. Treatment efficacy is determined from the reduction in systolic blood pressure, diastolic blood pressure and heart beats of the animal compared to the untreated control group.
Toxicity and Necropsy Examination. Animals were weighed daily throughout the experiment. The rats were examined every hour for overt signs of any adverse, drug-related side effects along with the changes in mucus membrane, snout and oral cavity, nasal discharge, watery eyes, rough coat, group and individual behavior, food and water intake, urination/defecation or any other observable symptoms and if any clinical manifestations.

In general, a drug study conducted on rats, the females should be a minimum of 120 g and males 140 g, even though the general suggestion is higher body weight. The variation in body weight of all animals within the trial were less than 5 g. The animals were weighed daily during the drug treatment period including weekends and holidays for the entire study period. The daily weighing is usually the best method to assess toxicity in a flexible dose schedule screening trial, and is an indication of onset of infection.

Acceptable toxicity for drugs in rats is usually defined as group's mean body-weight loss of less than 20% during the test, and not more than 10% toxic death among related animals. Other toxicities encountered in primary screening trials: neurologic (stupor, ataxia, peripheral neuropathy, splay-foot-walk, seizures, coma, spasms, tremors, unconscious lying on its side and so forth); respiratory problems; activity level (jumping, running, crouched, no movement, avoidance behavior); grooming or lack thereof, tissue damage, stomatitis; squealing; and animals look poorly.

In the necropsy examination, moribund animals were euthanized with carbon dioxide and the animals were examined for any changes in the natural orifices for any discoloration, discharge etc. A ventral incision was made from the peritoneal end to the thoracic end of the animal and the organs were examined under a dissection microscope, assessing the spleen size, appearance of liver, lungs, kidneys, GI tract, heart and other organs. This information is useful for deaths that occurred during drug treatment and is critical for identifying slightly delayed drug deaths.

F. Renin Toxicity Studies

A standard up-and-down test procedure as described in OEDC 425 was used to assess oral toxicity of compound 15 (Tristenonol aglycone) of the present invention. Five Sprague Dawley female rats were used and food was withheld from the animals the night prior to the test. Animals were weighed prior to treatment and animals were dosed with compound [15] of the present invention by intragastic intubation using a ball tip gavaging needle and syringe. Single animals were dosed sequentially at 48 h intervals and clinical observations were made at least twice daily on the day of dosing. The first animal was dosed at 2000 mg kg⁻¹, and since it survived the treatment, the remaining four animals were dosed sequentially. After dosing the animals were returned to their cages and supplied with feed and water ad libitum. Clinical observations were performed at least once a day for the first 30 min after dosing for 14 days. Animals were weighed at the end of the observation period and were sacrificed by carbon disoxide inhalation. Changes in weight were recorded and a gross necropsy was conducted on all animals scarficed at the end of the study. All gross pathological features were recorded.

An assessment of mutagencity of the Tristenonol aglycone [15] of the present invention was assessed using OECD 471 and related international and U.S. approved methods (e.g., Federal Register Vol. 61, Apr. 24, 1996 p. 18199). The Salmonella typhimurium and Escherichia coli reverse mutation assay (Ames Assay) was utilized to determine the potential of Tristenonol aglycone [15] of the present invention to induce reverse mutations in histidine (his⁻ to his⁺) and tryptophan (tryp⁻ to tryp⁺) genes in S. typhimurium and E. coli, respectively. Strains TA98 and TA100 of S. typhimurium were used in the absence of metabolic activation with both direct and preincubation methods for range finding. The test assays were conducted with four strains of S. typhimurium (TA98, TA100, TA1535, and TA1537) and one strain of E. coli (WP2 uvrA).

### Results

A. Renin Activity Inhibition

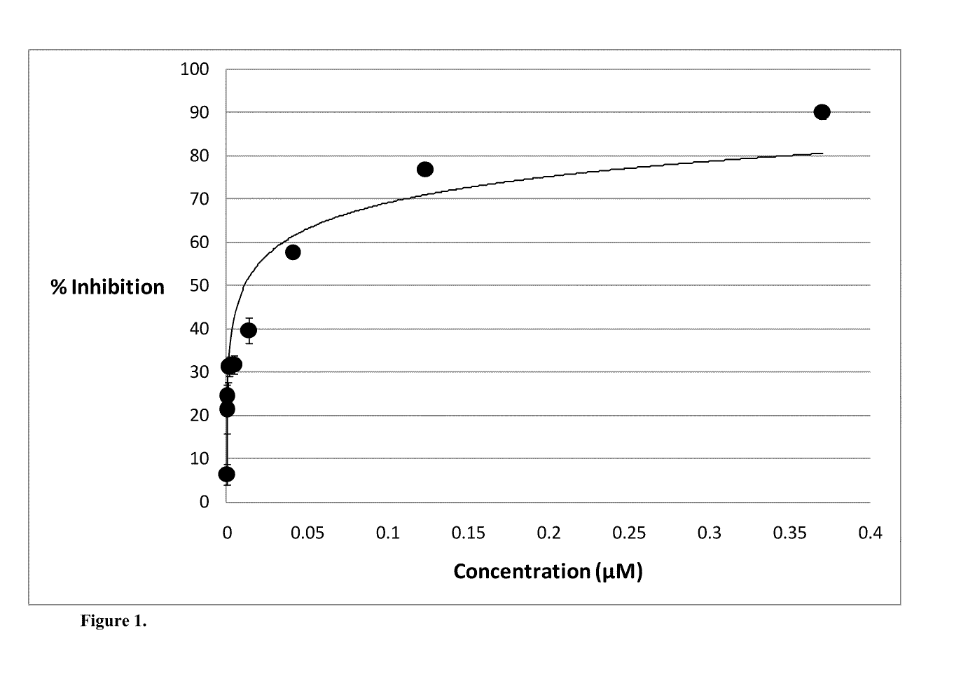

Compounds inhibit the Renin enzyme as evidenced using compound [15] of the present invention in a Renin inhibition assay as described above. Compound [15] achieved and IC₅₀ value of 10.2 μM and 100% inhibition was achieved at 2.5 mM based on the extrapolated line-of-best-fit (FIG. 1).

B. Absorption, Distribution, Metabolism, Excretion, and Toxicity (ADMET) Predictions

Based on the calculations, compounds of the present invention will be absorbed in the small intestine, are likely to pass through the blood brain barrier, and have low interactions with Cytochrome P450. Using similar molecular modeling tools, it was determined that compounds of the present invention are not mutagenic, based on AMES mutagenicity predictions, and they have a predicted rat oral LD₅₀ of 1.9 g Kg⁻¹ indicating that these compounds are not toxic.

C. Molecular Modeling

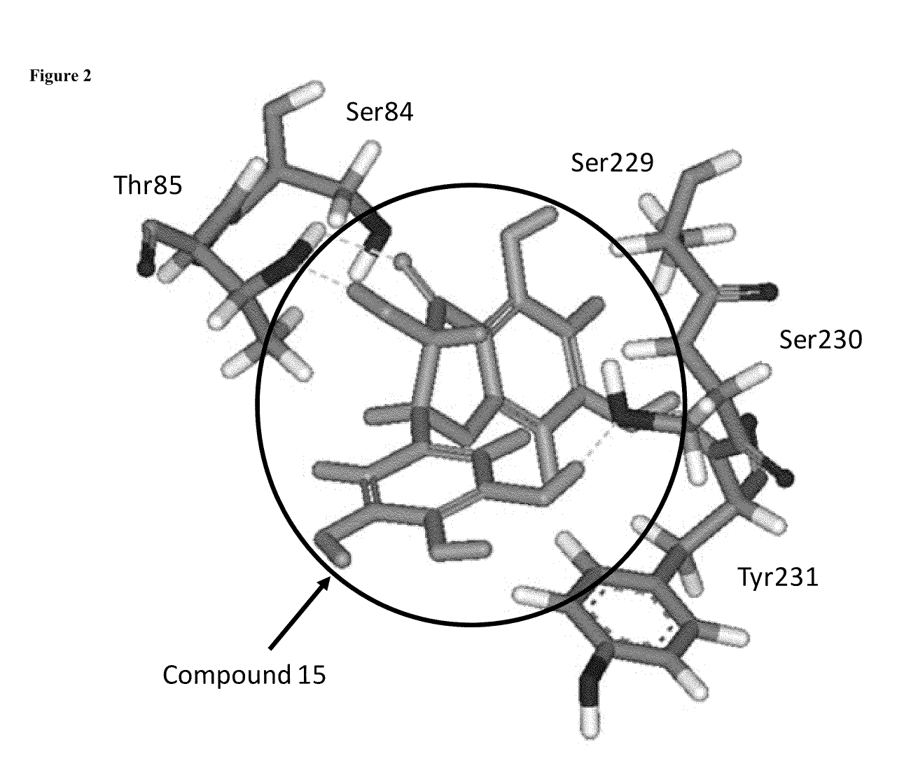

While not being bound by any particular theory, it is believed that the compounds of the present invention as exemplified by compound [15] of the present invention reveals high binding affinity between compound [15] and the binding site of the Renin enzyme. Multiple hydrogen bonds are formed between compound [15] and the amino acids Ser230, Tyr231, Ser84, and Thr85 present in the Renin binding site (FIG. 2).

D. Pharmacokinetic Analysis

The primary pharmacokinetic parameters for a compound of the present invention were determined from a 175-mg lozenge dose. The maximum serum concentration of compound [15] of 3.9±0.8 nmol L⁻¹ was achieved at 4 hours post-ingestion. The elimination rate constant for compound [15] is 0.026 nmol L⁻¹ min⁻¹ giving a serum half-life of 26 min. Additionally when 350 mg of a mixture of chemicals containing compound [15] was delivered as a liquid, the primary pharmacokinetic parameters changed. Here, the maximum serum concentration of compound [15] of 17.0 nmol L⁻¹ (only one subject) was achieved at 60 min post-ingestion. The elimination rate constant for compound [15] when delivered in liquid form is 0.101 nmol L⁻¹ min⁻¹ providing a serum half-life of 6.9 min.

E. Animal Studies

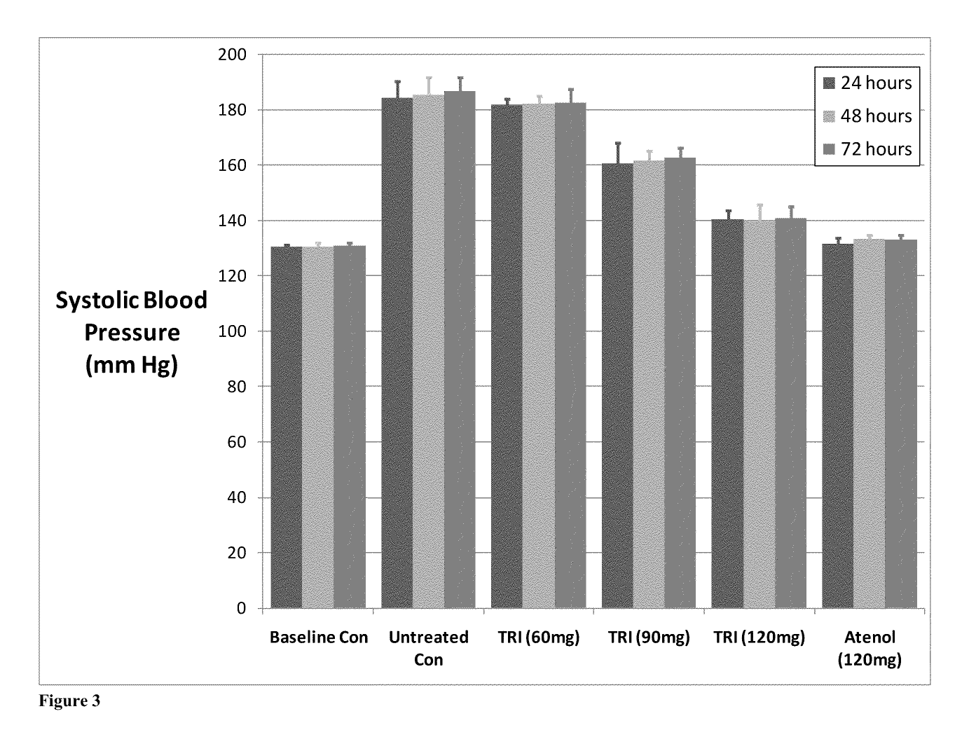

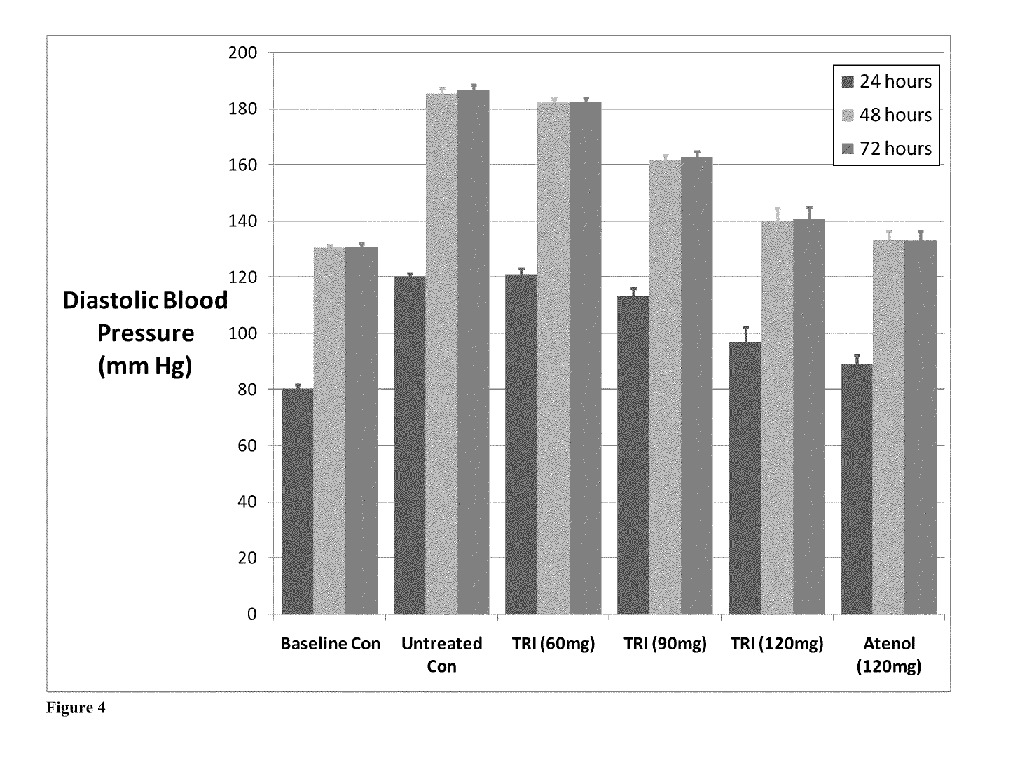

The response of Systoic blood pressure to control, experimental (Cmp 15, Tristenonol aglycone of the present invention), and a positive control (Atenol®) are shown in FIG. 3. The mean reduction of Systolic blood pressure at 24, 48, and 72 h after Tristenonol aglycone or Atenol® was administered to the rats treated with monocrotaline is provided. Monocrotaline induced 40-45% increase in Systolic blood pressure in injected rats (FIG. 3) and induced 49-52% increase in Diastolic blood pressure (FIG. 4).

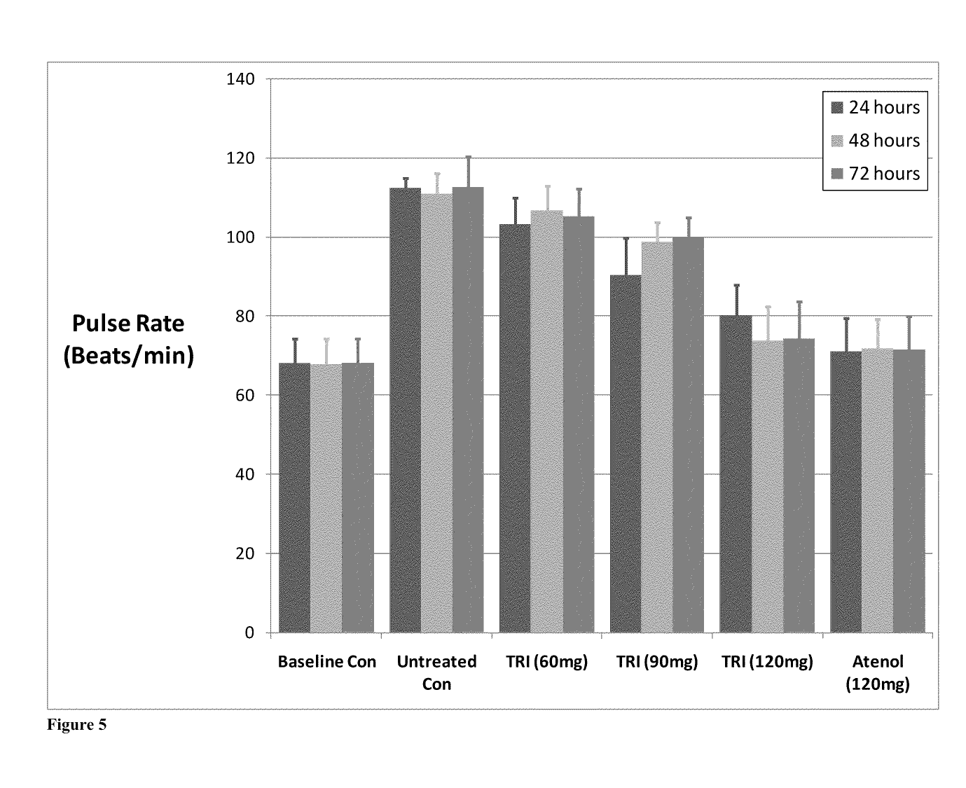

Monocrotaline caused a 60-70% increase in pulse rate in injected mice (FIG. 5). Post mortem examination of Groups 1-6 showed no observable lesions, and there were nonchanges in food and water intake and no changes in weight gain or loss. Further, urination and defecation was normal and group behavior was normal. Therefore, it was concluded that none of the groups showed any signs of treatment toxicity during the duration of the study or during initial post-mortem observations. The weight loss for those groups that were given the Tristenonol aglycone [Cmp 15] were within the marginal limits of animal study guidelines and the animals regained weight, comparable to the controls groups. The range of body weight loss observed in the compound [15] treated animals was between 1.47 and 1.7%. The weight loss was calculated from the single animal linear progression and was consistent within the permissible limits set forth, not to exceed 10% of the body weight.

The blood pressure both systolic and diastolic of animals receiving compound [15] Tristenonol aglycone and Atenol® were compared to untreated animals. The groups that received oral administration of compound [15] had reduced mean Systolic blood pressure. The response was dose-dependent with a 24-25% reduction in the 120 mg kg⁻¹ body weight treated rats, 12-13% reduction in the 90 mg kg⁻¹ body weight treated rats, and a 1-2% reduction in the 60 mg kg⁻¹ body weight treated rats compared to the untreated animals (FIG. 3). The group that received the Atenol® showed a 28% decrease in Systolic blood pressure compared with untreated animals.

The diastolic blood pressure was also reduced in dose-dependent manner in compound [15] treated rats with a 17-19% decrease in the 120 mg kg⁻¹ body weight treated rats, a 6-8% decrease in the 90 mg kg⁻¹ body weight treated rats, and a 0% decrease in the 60 mg kg⁻¹ body weight treated rats, compared to the untreated control (FIG. 4). The group that received the Atenol® showed a 24-25% decreased in Diastolic blood pressure compared with untreated animals.

Pulse rates were also reduced in compound [15] treated rats with a 28-34% reduction in the 120 mg kg⁻¹ body weight treated rats, with all-20% reduction in the 90 mg kg⁻¹ body weight treated rats, and a 4-8% reduction in the 60 mg kg⁻¹ body weight treated rats, compared to untreated controls (FIG. 5). Mean reduction in pulse rates of Atenol® administered animals was 35-36.38% compared to untreated control. Compound [15] of the present invention caused a dose-dependent decrease in blood pressure and heart beats in the monocrotaline-induced hypertension rat model.

Post mortem investigation conducted on animals found no lesions in the lungs including the internal organs such as brain, liver, heart, diaphragm, viscera and no other lesions were observed in animals treated with compound [15]. There were no observable adverse side effects noted in the treated animals.

F. Toxicity and Mutagenicity Studies

All animals gained weight by the end of the oral toxicity studies and no unusual necropsy was found in any of the treated animals. The animals dosed at 2000 mg kg⁻¹ did not show any signs of toxicity during the study. The estimated LD₅₀ for compound [15] of the present study is >2000 mg kg⁻¹ consistent with the ADMET predicted oral toxicity of >1900 mg kg⁻¹.

The range finding evaluations for the Ames test revealed that the three highest concentrations of compound [15] (5000, 1667, and 555 μg per plate) were cytotoxic only in TA98 but not in TA100. Therefore, the Ames assays were conducted over the full concentration range. The Ames tests showed that compound [15] of the present invention did not increase the frequency of revertants at any concentration as compared to negative controls. This data is consistent with the ADMET predictions for compound [15] of the present invention that indicate no mutagenicity.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

### INCORPORATION BY REFERENCE

All publications, patents, and patent applications cited herein are hereby incorporated by reference in their entirety.

We claim:

1. A method of treating a cardiovascular or renal condition in a subject in need thereof comprising administering to the subject a therapeutically effective amount of a compound of formula I: 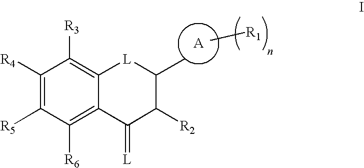
or a pharmaceutically acceptable salt thereof,
wherein, independently for each occurrence:
R₁ represents alkoxy, alkenyloxy, alkynyloxy, aryloxy, arylalkyloxy, hydroxy, —OC(O)—R₇, alkyl, alkenyl, alkynyl, acetyl, formyl, halide, cyano, nitro, SH, amino, amido, sulfonyl, or sulfonamido;
R₂ represents —OH or 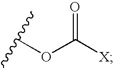
R₃, R₄, R₅, and R₆ represent H, hydroxy, alkoxy, alkenyloxy, alkynyloxy, aryloxy, aralkyloxy; —OC(O)—R₇, alkyl, alkenyl, alkynyl, aralkyl, acetyl, formyl, halide, cyano, nitro, SH, amino, amido, sulfonyl, or sulfonamido;
R₇ represents H, alkyl, alkenyl, alkynyl, cycloalkyl, aryl, arylalkyl or a carbohydrate;
A represents an aryl group;
L represents O, S, or NR;
R represents H, hydroxy, alkyl, alkenyl, alkynyl, aralkyl, acetyl, formyl, or sulfonyl;
X represents a carbohydrate, a cycloalkyl, or a cycloalkenyl group; and
n represents an integer from 1 to 5, inclusive;
wherein any of the aforementioned alkoxy, alkenyloxy, alkynyloxy, aryloxy, aralkyloxy, alkyl, alkenyl, alkynyl, cycloalkyl, cycloalkenyl, aryl and aralkyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

2. The method of claim 1, wherein, independently for each occurrence:
R₁ represents H, alkoxy, aryloxy, aralkyloxy, hydroxy, —OC(O)—R₇, alkyl, acetyl, formyl, or halide;
R₂ represents 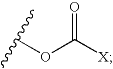
R₃, R₄, R₅, and R₆ represent H, alkoxy, aryloxy, aralkyloxy; —OC(O)—R₇, alkyl, aralkyl, acetyl, formyl, or halide;
R₇ represents H, alkyl, aryl, or arylalkyl;
A represents an aryl group;
L represents O;
X represents a carbohydrate, a cycloalkyl, or a cycloalkenyl group; and
n represents an integer from 1 to 5, inclusive;
wherein any of the aforementioned alkoxy, alkenyloxy, alkynyloxy, aryloxy, aralkyloxy, alkyl, alkenyl, alkynyl, aryl, aralkyl, cycloalkyl and cycloalkenyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

3. The method of claim 1, wherein the carbohydrate is selected from the group consisting of a monosaccharide, a disaccharide, an oligosaccharide, and a polysaccharide.

4. The method of claim 1, wherein R₂ is —OH.

5. The method of claim 1, wherein L is O.

6. The method of claim 1, wherein R₃, R₄, R₅ and R₆ are each independently H or hydroxy, wherein at least two of R₃, R₄, R₅ and R₆ are hydroxy.

7. The method of claim 1, wherein R₁ is hydroxy, and n is equal to 2 or 3.

8. The method of claim 1, wherein A is a benzene ring.

9. The method of claim 1, wherein X is a carbohydrate.

10. The method of claim 1, wherein X is a cycloalkyl or cycloalkenyl group; and wherein the cycloalkyl or cycloalkenyl group is substituted with 1 to 3 hydroxy groups.

11. A method of treating a cardiovascular or renal condition in a subject in need thereof comprising administering to the subject a therapeutically effective amount of a compound of formula Ia: 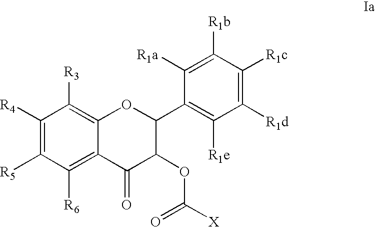
or a pharmaceutically acceptable salt thereof,
wherein, independently for each occurrence:
R₁ₐ, R_{1b}, R_{1c}, R_{1d}, R₁ₑ represent H, hydroxy, alkoxy, aralkyloxy, or aryloxy;
R₃, R₄, R₅, and R₆ represent H, hydroxy, alkoxy, aryloxy, or aralkyloxy; and
X represents a carbohydrate, a cycloalkyl, or a cycloalkenyl group;
wherein any of the aforementioned alkoxy, aryloxy, aralkyloxy may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

12. The method of claim 11, wherein: independently for each occurrence:
R₁ₐ, R_{1b}, R_{1c}, R_{1d}, and R₁ₑ represent H, hydroxy, alkoxy, aralkyloxy, or aryloxy; provided that at least two of R₁ₐ, R_{1b}, R_{1c}, R_{1d}, and R₁ₑ are hydroxy;
R₃, R₄, R₅, and R₆ represent H, hydroxy, alkoxy, aryloxy, or aralkyloxy, provided that at least two of R₃, R₄, R₅, and R₆ are hydroxy; and
X is carbohydrate, cycloalkyl, or cycloalkenyl;
wherein any of the aforementioned alkoxy, aryloxy, aralkyloxy, cycloalkyl, or cycloalkenyl may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

13. The method of claim 11, wherein:
R₁ₐ, R_{1b}, R_{1c}, R_{1d}, and R₁ₑ represent H or hydroxy, and three of R₁ₐ, R_{1b}, R_{1c}, R_{1d}, and R₁ₑ are hydroxy.

14. The method of claim 11, wherein:
R₃, R₄, R₅, and R₆ represent H or hydroxy, and two of R₃, R₄, R₅, and R₆ are hydroxy.

15. The method of claim 11, wherein:
X is a carbohydrate selected from the group consisting of a monosaccharide, a disaccharide, an oligosaccharide, and a polysaccharide.

16. The method of claim 11, wherein:
X is a carbohydrate selected from the group consisting of arabinose, lyxose, ribose, rhamnose, deoxyribose, xylose, ribulose, xylulose, allose, altrose, galactose, glucose, gulose, idose, mannose, talose, fructose, psicose, sorbose, tagatose, sucrose, lactose, maltose, trehalose or cellobiose, raffinose, maltodextrin, cyclodextrin, starch, glycogen, dextran, and cellulose.

17. The method of claim 11, wherein X is rhamnose.

18. The method of claim 11, wherein X is a cycloalkyl or cyloalkynyl group, wherein the cycloalkyl or cycloalkenyl group is substituted with one to three hydroxy groups.

19. A method of treating or preventing a cardiovascular or renal condition in a subject in need thereof comprising administering to the subject a therapeutically effective amount of a compound of formula Ib: 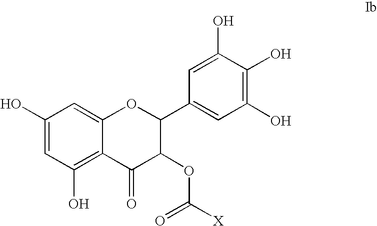
or a pharmaceutically acceptable salt thereof,
wherein X represents a carbohydrate, a cycloalkyl, or a cycloalkenyl, wherein the cycloalkyl or cycloalkenyl may be substituted with one to three hydroxy groups.

20. The method of claim 19, wherein X is a carbohydrate selected from the group consisting of arabinose, lyxose, ribose, rhamnose, deoxyribose, xylose, ribulose, xylulose, allose, altrose, galactose, glucose, gulose, idose, mannose, talose, fructose, psicose, sorbose, tagatose, sucrose, lactose, maltose, trehalose, cellobiose, raffinose, maltodextrin, cyclodextrin, starch, glycogen, dextran, and cellulose.

21. The method of claim 19, wherein X is a cyclohexyl or cyclohexenyl substituted with 1 to 3 hydroxy groups.

22. The method of claim 21, wherein X is:

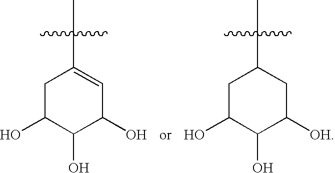

23. A method of treating or preventing a cardiovascular or renal condition in a subject in need thereof comprising administering to the subject a therapeutically effective amount of a compound selected from the group consisting of: 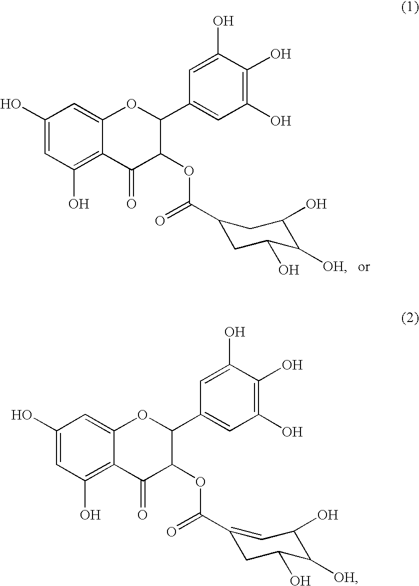
and pharmaceutically acceptable salts thereof.

24. A method of treating or preventing a cardiovascular or renal condition in a subject in need thereof comprising administering to the subject a therapeutically effective amount of a compound of formula Ic: 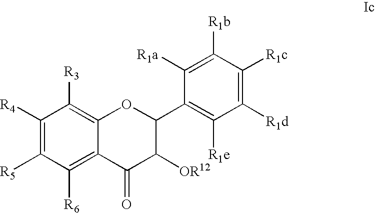
or a pharmaceutically acceptable salt thereof,
wherein, independently for each occurrence:
R₁ₐ, R_{1b}, R_{1c}, R_{1d}, R₁ₑ represent H, hydroxy, alkoxy, aralkyloxy, or aryloxy;
R₃, R₄, R₅, and R₆ represent H, hydroxy, alkoxy, aryloxy, or aralkyloxy; and
R₁₂ represents H, hydroxy, alkoxy, aralkyloxy, or aryloxy;
wherein any of the aforementioned alkoxy, aryloxy, aralkyloxy may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

25. The method of claim 24, wherein R₁ₐ, R_{1b}, R_{1c}, R_{1d}, and R₁ₑ represent H, hydroxy, alkoxy, aralkyloxy, or aryloxy; provided that at least two of R₁ₐ, R_{1b}, R_{1c}, R_{1d}, and R₁ₑ are hydroxy;
R₃, R₄, R₅, and R₆ represent H, hydroxy, alkoxy, aryloxy, or aralkyloxy, provided that at least two of R₃, R₄, R₅, and R₆ are hydroxy; and
wherein any of the aforementioned alkoxy, aryloxy, aralkyloxy, cycloalkyl, or cycloalkenyl may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

26. The method of claim 25, wherein:
R₁ₐ, R_{1b}, R_{1c}, R_{1d}, and R₁ₑ represent H or hydroxy, and three of R₁ₐ, R_{1b}, R_{1c}, R_{1d}, and R₁ₑ are hydroxy.

27. The method of claim 25, wherein:
R₃, R₄, R₅, and R₆ represent H or hydroxy, and two of R₃, R₄, R₅, and R₆ are hydroxy.

28. The method of claim 24, wherein R₁₂ is OH.

29. A method of treating or preventing a cardiovascular or renal condition in a subject in need thereof comprising administering to the subject a therapeutically effective amount of the following compound: 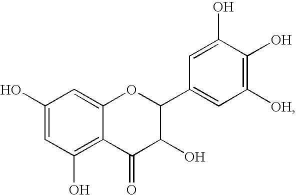
or a pharmaceutically acceptable salt thereof.

30. The method of claim 1, wherein the method inhibits renin activity.

31. The method of claim 1, wherein the subject has a cardiovascular disease.

32. The method of claim 1, wherein the method maintains electrolyte homeostasis.

33. The method of claim 1, wherein the cardiovascular condition selected from the group consisting of hypertension, severe hypertension, pulmonary hypertension (PH), malignant hypertension, isolated systolic hypertension, familial dyslipidemic hypertension, high blood pressure, atherosclerosis, unstable coronary syndrome, congestive heart failure, myocardial infarction, cardiac hypertrophy, cardiac fibrosis, cardiomyopathy postinfarction, unstable coronary syndrome, diastolic dysfunction, complications resulting from diabetes, diseases of the coronary vessels, elevated total cholesterol, low LDL cholesterol, peripheral vascular disease (PVD), peripheral artery disease (PAD), peripheral venous disorders, coronary arterial disease (CAD), restenosis following angioplasty, raised intra-ocular pressure, glaucoma, abnormal vascular growth, hyperaldosteronism, cerebrovascular diseases, metabolic disorder (Syndrome X), atrial fibrillation (AF), vascular inflammation, vasculitides or closure, aneurysm, angina, and restenosis of dialysis access grafts.

34. The method of claim 1, wherein the renal condition is selected from the group consisting of renal failure, chronic kidney disease, renoprotection, reduction of proteinuria, glomerulonephritis, nephrotic syndrome, renal fibrosis, acute interstitial nephritis (AIN), acute tubular nephritis (ATN), acute tubulo-interstitial nephritis, polycystic kidney disease (PKD), endothelial dysfunction, and microalbuminuria,

35. The method of claim 1, wherein the compound is administered in combination with one or more pharmaceutically acceptable carriers.

36. The method of claim 1, wherein the compound is administered in combination with at least one additional active agent selected from the group consisting of an angiotensin II receptor antagonist, an ACE inhibitor, a calcium channel blocker, an HMG-CO-A reductase inhibitor, an aldosterone synthase inhibitor, an aldosterone antagonist, an ACE/NEP inhibitor, a beta-blocker, an endothelin antagonist, and a diuretic.

37. The method of claim 1, wherein the subject is a mammal.

38. The method of claim 37, wherein the subject is a primate.

39. The method of claim 38, wherein the subject is human.
